(19) United States
(12) Patent Application Publication
(10) Pub. No.: US 2009/0131953 A1
(43) Pub. Date: May 21, 2009

(54) Flexible Intraocular Implant Injector

(76) Inventors: Nicolas Quintin, Pologne (FR); Cyrille Vinchon, Chilly (FR); Jean-Marc Andre, La Motte Servolex (FR)

Correspondence Address: KENYON & KENYON LLP, 1500 K STREET N.W., SUITE 700, WASHINGTON, DC, 20005, US

(21) Appl. No.: 11/795,336

(22) PCT Filed: Jan. 20, 2006
(86) PCT No.: PCT/FR2006/050030

§ 371 Date: Nov. 15, 2007

(30) Foreign Application Priority Data
Jan. 21, 2005 (FR) 0500633

(51) Int. Cl. A61F 9/007

(52) U.S. Cl. 606/107

(57) ABSTRACT

The injector for injecting a flexible intraocular implant comprises: a channel (14) having a main portion (18, 30) of diameter D1, an open end portion (34) of diameter D2 (with D2<D1), and a transition portion (32); a rigid piston (16) movable along the channel to push the folded implant in the channel, and a substantially circularly symmetrical thrust part (40) made of a material that is hydrophobic and elastically deformable, said thrust part having an outside diameter D3 not less than D1, said part (40) being placed in said channel between the folded implant (1) and the end of said piston (16) without being mechanically connected to said piston. When the implant is pushed in the channel by the piston, said thrust part can deform elastically to penetrate in part into the end portion of the channel, thereby expelling the implant from the channel, and remaining jammed in said end portion of the channel when the piston is withdrawn.

This is a 371 national phase application of PCT/FR2006//050030 filed 20 Jan. 2006, claiming priority to French Patent Application No. 05300633 filed 21 Jan. 2005, the contents of which are incorporated herein by reference.

### BACKGROUND OF THE INVENTION

The present invention provides an injector for injecting a flexible intraocular implant.

Such flexible implant injectors are described in particular in patent applications PCT/FR01/01510 and PCT/FR01/02994 in the name of the supplier Corneal Industrie.

Flexible intraocular implants are implants having at least the optical portion made of a deformable material such as a hydrophilic or hydrophobic acrylic. The optical portion and also the haptic portion of such implants can be folded or rolled up in such a manner that while in this state the implant can be inserted into the eye through an incision of small size, typically of the order of 3 millimeters (mm).

To facilitate the work of the surgeon, devices known as flexible implant injectors have been developed that make it possible firstly to fold the implant, and secondly to place the folded implant inside the eye by means of a cannula that is inserted into the incision made in the wall of the eye.

The injector essentially comprises a folding chamber in which the non-folded implant can be placed, and in which, by moving a mechanical member, the implant is properly rolled up or folded. The injector also has an internal channel forming a cannula in which the folded implant can be moved with the help of a piston up to the open end of the cannula which is placed inside the eye. When the piston reaches the end its stroke, the implant is ejected into the inside of the eye in the appropriate location, and it returns spontaneously to its initial shape.

To further decrease the size of the incision made in the wall of the eye, implant injectors have been developed in which the end of the cannula disposed inside the eye has a diameter that is further reduced relative to that of the folding chamber. Thus, by pushing the implant that has already been folded along the channel of the cannula, the outside diameter of the folded implant is further reduced. That type of injector raises certain problems concerning the action of the piston on the folded implant at the end of its stroke.

At least the end of the injector inserted into the patient's eye needs to be sterilized after use. To avoid problems and risks associated with sterilization being performed more or less thoroughly, injectors have been put on the market that are made out of plastics materials that are not reusable since they cannot be sterilized effectively.

When such an injector is used under such circumstances, it is important to make the injector effectively and definitively non-reusable after it has been used for the first time.

### SUMMARY OF THE INVENTION

An object of the present invention is to provide a flexible implant injector that makes it possible firstly to fold the implant so as to give it dimensions that are further reduced, and secondly that is effectively non-reusable after it has been used for a first time.

According to the invention, to achieve this object, the flexible implant injector comprises a channel having a main portion of diameter D1, an open end portion of diameter D2 (with D2<D1), and a transition portion of substantially frustoconical shape connecting said main portion to said end portion, with a rigid piston movable in the channel to push the folded implant in the channel.

Said injector is characterized in that it further comprises a substantially circularly symmetrical thrust part made of a material that is hydrophobic and elastically deformable, said thrust part having, in the absence of stress, an outside diameter D3 not less than D1, said part being placed in said channel between the folded implant and the end of said piston without being mechanically connected to said piston, whereby, when the implant is pushed along the channel by the piston, said thrust part can, by elastic deformation, penetrate in part into the end portion of the channel, thereby expelling the implant from the channel, said thrust part remaining jammed in said end portion of the channel on the piston being withdrawn, thereby making the injector non-reusable.

The term “not mechanically connected to the piston” should be understood as indicating that there are no mechanical connection means between the end of the piston and the thrust part, whether by adhesive, by mutual engagement, by overmolding, etc. The thrust part is completely free relative to the piston and it is merely pushed by the piston, merely by contact between the end of the piston and the posterior face of the thrust part.

It will be understood that because the end of the injector channel presents a diameter that is smaller than the diameter of its main portion, which corresponds to the diameter of the rolling or folding chamber, the implant becomes more folded, so the size of the implant is even smaller. Furthermore, because of the presence of the elastically deformable thrust part, the implant can be pushed effectively, including in the small diameter terminal portion of the channel of the injector, thus ensuring that the implant is properly ejected into the inside of the patient's eye. In addition, it will be understood that because the thrust part is under no circumstances mechanically connected to the piston, when the piston reaches the end of its stroke, the deformable thrust part has been inserted into the smaller-diameter end of the channel of the injector, and said part remains jammed therein when the surgeon proceeds to withdraw the piston. The jamming of the thrust part in the end of the channel of the injector makes the injector completely non-reusable.

Finally, because the thrust part is not mechanically connected to the piston, the radial deformation of the thrust part is uniform and regular.

In a preferred embodiment, the end of the thrust part for co-operating with the end of the piston has a diameter D4>D1, and the other end of the thrust part for co-operating with the folded implant has a diameter D5 substantially equal to D1, whereby the radial elastic deformation of the first end of the thrust part creates a coefficient of friction between itself and the inside wall of the channel.

It will be understood that because the posterior end of the thrust part, i.e. its end facing towards the piston, has an outside diameter that is perceptibly greater than the diameter of the inside wall of the channel, this posterior end is slightly compressed. This ensures that the inside wall of the channel is scraped even if it has certain amount if roughnesses. Such scraping serves to avoid the haptic loops jamming between the thrust part and the inside wall of the channel. In addition, this compression of the posterior portion of the thrust part ensures a constant coefficient of friction between the thrust part and the inside wall of the channel, thereby making the surgeon's work easier for obtaining regular displacement of the implant along the channel, in particular during the final stage of thrust serving to eject the implant from the end of the injector.

### BRIEF DESCRIPTION OF THE DRAWINGS

Other characteristics and advantages of the invention appear better on reading the following description of a preferred embodiment of the invention given by way of non-limiting example:

FIG. 1 is an overall perspective view of a flexible implant injector;

FIGS. 2A to 2D are longitudinal section views of the anterior portion of the injector, showing how the injector is used; and

FIG. 3 is a side view of the thrust part.

### DETAILED DESCRIPTION

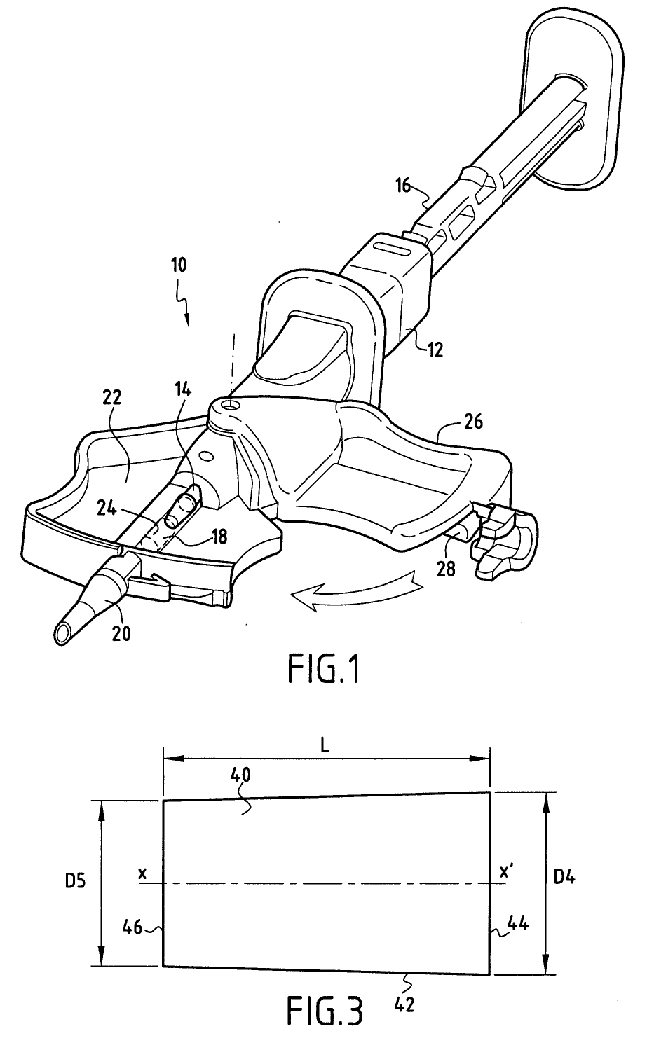

With reference to FIG. 1, there follows an overall description of a flexible implant injector of the type in question. The injector 10 comprises a body 12 defining a channel 14. The channel 14 has a posterior portion 15 for guiding a piston 16, an intermediate portion 18 constituting a chamber for folding the flexible implant, and an anterior portion 20 constituting a cannula. The folding chamber 18 has a stationary portion 22 defining a first jaw half 24, and a moving portion 26 defining a second jaw portion 28 for folding purposes.

When a non-folded implant is put into place in the folding chamber 18, and the moving portion 26 is moved towards the stationary portion 22, the flexible implant folds progressively such that at the end of this operation the implant is folded within the folding chamber 18.

Once the flexible implant has been folded inside the chamber 18, the piston 16 is used to push the folded implant into the cannula 20 that has been inserted into an incision formed in the eye of the patient so as to insert the implant into the patient's eye.

The above description describes to the general organization of a known flexible implant injector.

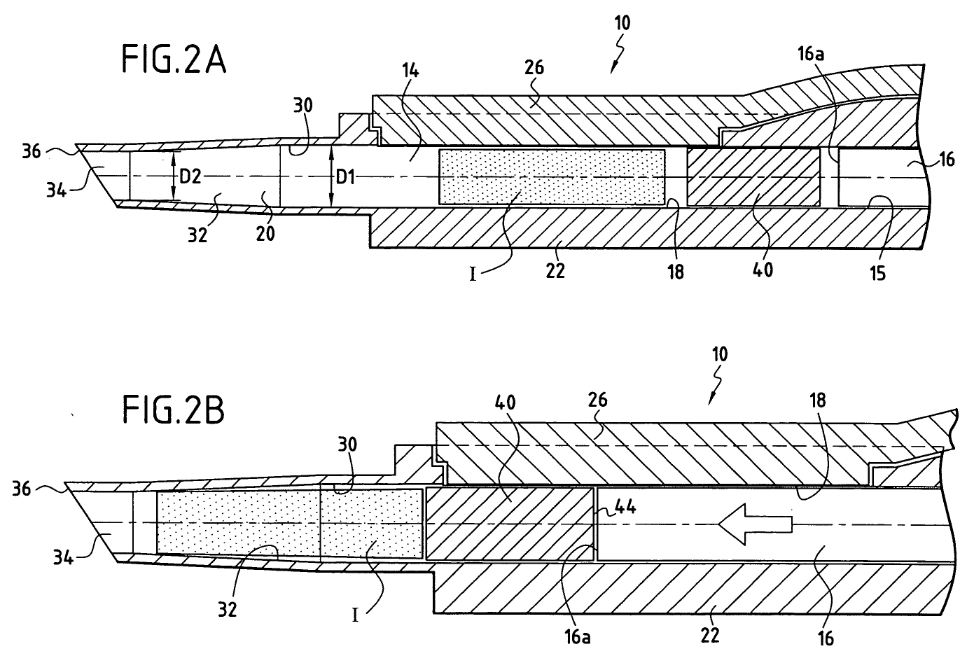

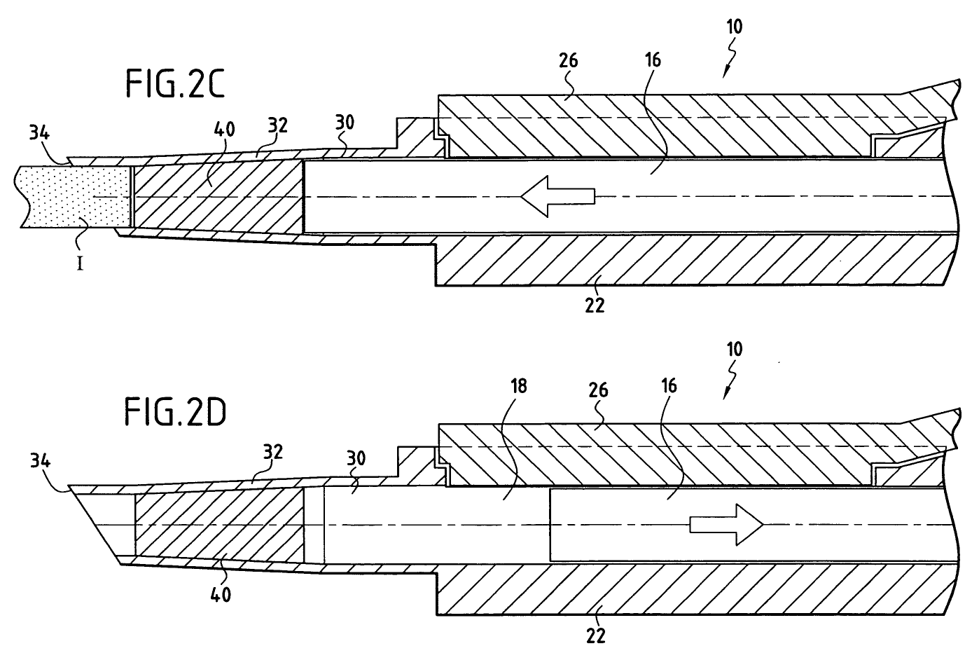

Reference is now made to FIGS. 2A to 2D, and to FIG. 3, while describing the particular type of implant injector that constitutes the subject matter of the present invention.

In FIG. 2A, there can be seen the anterior portion of the implant injector 10. There can be seen the folding chamber 18 defined by the stationary jaw 22 and the moving jaw 26. There can also be seen the channel 14 which is constituted by a posterior portion 15 for guiding the piston 16, by the folding chamber 18, and by an anterior portion forming a cannula 20.

As shown in FIG. 2A, the anterior portion 20 of the channel has a first fraction 30 connected to the outlet from the folding chamber and presenting a diameter D1 which is the same as the diameter of the main portion of the channel 14, an intermediate transition fraction 32 of inside diameter varying from D1 to D2, where D2 is less than D1, and a terminal portion 34 of diameter D2. The terminal portion 34 preferably has a chamfered end edge 36.

If the entire channel 14 is considered, it can be seen firstly that there is a fraction 30 of diameter D1 corresponding to the diameter of the folding chamber and thus to the diameter of the folded implant, and then a fraction 32 of inside diameter that tapers to the value D2, followed by the terminal portion of diameter D2. It will readily be understood that by pushing the folded implant I with the help of the piston 16 in the channel 14, the outside rolled-up diameter of the implant is caused to change from D1 to D2.

In the invention, in an initial position within the channel 14, a thrust part 40 is interposed between the end 16a of the piston 16 and the folded implant I as initially placed in the roll-up chamber. The thrust exerted by the piston 16 is thus transmitted to the folded implant I via the thrust part 40.

With reference to FIG. 3, there follows a description in greater detail of the thrust part 40. The thrust part 40 presents a side face 42 that is circularly symmetrical about the longitudinal axis X, X′, an anterior face 46 for coming into contact with the folded implant I, and a posterior face 44 for coming into contact with the end 16a of the piston 16. The end faces 44 and 46 are substantially orthogonal to the longitudinal axis X, X′. In addition, the diameter of the anterior face 46, referenced D5, is substantially equal to the diameter D1 of the main portion of the channel 14. In contrast, the posterior face 44 has a diameter D4 that is greater than the diameter D1. In addition, the length L of the thrust part 40 is substantially equal to the length of the transition fraction 32 of the channel.

The thrust part 40 is made, preferably entirely, out of a material that is elastically deformable and that is hydrophobic. A particularly suitable material is silicone.

It will be understood that when the thrust part 40 is placed inside the channel 14, its posterior end of diameter D4 is compressed. This compression means that while it is moving in the channel 14, the thrust part scrapes the inside face of the channel and creates a coefficient of friction between itself and the wall of the channel.

In addition, since the thrust part is hydrophobic, its dimensions are not modified by the liquid present in the injector.

Preferably, the diameter D4 of the posterior portion of the thrust part 40 lies in the range 1.15 times to 1.25 times the diameter D5 of its anterior face.

Consequently, the outside diameter D3 of the thrust part 40 is at least equal to D5, i.e. D1.

With reference now to FIGS. 2A to 2D, there follows a description of how the implant injector is used by a surgeon.

In FIG. 2A, the already-folded implant I is shown symbolically in the folding chamber. In this initial position, the thrust part 40 is located at the inlet of the folding chamber 18, in the channel 14. Because of its generally frustoconical shape, the thrust part is held in place in the channel by its posterior portion being compressed. When the surgeon exerts thrust on the piston 16, the end 16a thereof comes into contact with the posterior face 44 of the thrust part 40 and causes it to move into the folding chamber and then into the first portion 30 of the channel. When the thrust part 40 reaches the transition zone 32 of the channel, the part can pass easily in spite of its tapering diameter, because the part deforms radially in elastic manner. This is shown in FIG. 2B. The surgeon continues to exert thrust on the piston 16 so that the thrust part 40 penetrates into the end of the channel, thereby causing the initially folded implant to be ejected in controlled manner into the patient's eye. Furthermore, the fact that the thrust part 14 is compressed to a relatively great extent in the end portion 32, 34 of the channel ensures that the thrust part 40 remains jammed in the channel when the surgeon proceeds to withdraw the piston 16.

It can thus be understood that by means of the thrust part 40 jamming in the end of the channel of the implant injector, the injector cannot under any circumstances be reused for putting another flexible implant into place.

In a preferred embodiment, the diameter D1 of the main portion of the channel is substantially equal to 2 mm, the diameter D2 at the end of the channel is equal to 1.5 mm, the diameter D5 at the leading end of the thrust part 40 is equal to 2 mm, while its posterior end 44 has a diameter D4 equal to 2.2 mm in the absence of any stress. In addition, the length L of the thrust part 40 is equal to 4 mm.

It should be added that because of the compression of the posterior portion of the thrust part in the channel, the resulting coefficient of friction enables the surgeon to exert thrust on the piston 16 that is fully controlled, and thus to cause the folded implant in the channel to move in regular and non-jerky manner, thereby avoiding any untimely ejection of the implant into the inside of the patient's eye.

It should also be added that because the anterior end 16a of the piston 16 has a diameter substantially equal to that of the channel, risks of the deformed thrust part 40 jamming between the piston and the inside wall of the channel are avoided. Similarly, the fact that the anterior face 46 of the thrust part that is in contact with the folded implant has a diameter that is substantially equal to that of the channel avoids any risk of the haptic loops of the flexible implant jamming between the side wall of the thrust part 40 and the inside wall of the channel 14.

1.-8. (canceled)

9. An injector for injecting a flexible intraocular implant, the injector comprising:
a channel having a main portion of diameter D1, an open end portion of diameter D2 (with D2<D1), and a transition portion of substantially frustoconical shape for connecting said main portion to said end portion;
a rigid piston movable in the channel to push the folded implant in the channel, and:
a thrust part that is substantially circularly symmetrical, and made of a material that is hydrophobic and elastically deformable, said thrust part having, in the absence of applied stress, an outside diameter D3 not less than D1, said part being placed in said channel between the folded implant and the end of said piston without being mechanically connected to said piston, whereby, when the implant is pushed along the channel by the piston, said thrust part can, by elastic deformation, penetrate in part into the end portion of the channel, thereby expelling the implant from the channel, said thrust part remaining jammed in said end portion of the channel on the piston being withdrawn, thereby making the injector non-reusable.

10. An injector according to claim 9, wherein the end of said thrust part for co-operating with the end of the piston has a diameter D4>D1, and the other end of the thrust part for co-operating with the folded implant has a diameter D5 substantially equal to D1, whereby the radial elastic deformation of the first end of the thrust part creates a coefficient of friction between itself and the inside wall of the channel.

11. An injector according to claim 10, wherein the diameter D4 of the first end of the thrust part lies in the range 1.15 times to 1.25 times the diameter D5 of the second end.

12. An injector according to claim 9, wherein said thrust part is made entirely out of silicone.

13. An injector according to claim 9, wherein the diameter D3 of said piston is substantially equal to D1.

14. An injector according to claim 9, wherein the axial length of said thrust part is substantially equal to the length of the transition portion of the channel.

15. An injector according to claim 9, wherein the face of the first end of the thrust part is substantially plane and orthogonal to the longitudinal axis of the thrust part.

16. An injector according to claim 9, wherein said thrust part is made entirely out of said elastically deformable hydrophobic material.
